(19) United States
(12) Patent Application Publication
(10) Pub. No.: US 2009/0233893 A1
(43) Pub. Date: Sep. 17, 2009

(54) Methods of Dissolving Beta-Sheet Proteins and Uses Thereof

(76) Inventor: Laura Jeanne Juszczak, New York, NY (US)

Correspondence Address: Alan D Miller;Amster Rothstein & Ebenstein, 90 Park Avenue, New York, NY, 10016, US

(21) Appl. No.: 12/226,340

(22) PCT Filed: May 22, 2007
(86) PCT No.: PCT/US07/12244

§ 371 Date: Mar. 26, 2009

Related U.S. Application Data
(60) Provisional application No. 60/802,972, filed on May 24, 2006.

(51) Int. Cl. A61K 31/555; C07K 14/47; C07F 1/08; A61K 31/30; C07F 1/02; A61P 25/28; A61K 31/135; A61K 31/131

(52) U.S. Cl. 514/186; 530/324; 546/10; 514/185; 514/499; 530/345; 564/305; 564/463; 548/404; 548/402; 514/646; 514/671

(57) ABSTRACT

The application discloses metallized chelator complexes and uses of metallized chelator complexes for dissolving β-sheet proteins and reducing formation of β-sheet proteins, where the metallized chelator complex comprises a metal ion chelator and a metal ion.

### CROSS-REFERENCE TO RELATED APPLICATION

This application claims priority of U.S. Provisional Patent Application No. 60/802,972, filed May 24, 2006, the content of which is incorporated by reference.

### STATEMENT OF GOVERNMENT SUPPORT

The invention disclosed herein was made with U.S. Government support under National Institutes of Health grant number 5POI HL071064. Accordingly, the U.S. Government has certain rights in this invention.

### FIELD OF THE INVENTION

The application discloses metallized chelator complexes and uses of metallized chelator complexes for dissolving or solubilizing β-sheet proteins and reducing formation of β-sheet proteins.

### BACKGROUND OF THE INVENTION

Throughout this application various publications are referred to in parentheses. Citations for these references may be found at the end of the specification immediately preceding the claims. The disclosures of these publications are hereby incorporated by reference in their entireties into the subject application to more fully describe the art to which the subject application pertains.

The Beta (β) sheet structure of proteins results from hydrogen bonding between polypeptide chains of the protein. Solid accumulations of beta-sheet proteins (generally called amyloids) are common in a number of degenerative diseases, such as Alzheimer's disease, Creutzfeldt-Jakob disease and hereditary cerebral amyloid angiopathy. Solid proteins, often fibrogenic and β-sheet in structure, are associated with disease progression. Metal chelators have been utilized for in vivo studies of amyloid dissolution efficacy (Dedeoglu et al., 2004; Sigurdsson et al., 2003), most notably, for Alzheimer's disease, where copper, iron and zinc cations have been identified in amyloid plaques from diseased brain cross sections. Similarly, ‘lithium,’ i.e. lithium salts such as lithium chloride, has been found to inhibit the enzyme glycogen synthase kinase-3α, which is involved in processing two Alzheimer's disease amyloid-forming proteins, tau and amyloid-beta (Aβ), and therefore reduces amyloid plaque and neurofibrillary tangle formation (Alvarez et al., 1999; Phiel et al., 2003).

### SUMMARY OF THE INVENTION

The present invention provides methods of dissolving β-sheet proteins comprising contacting a β-sheet protein with a metallized chelator complex in an amount sufficient to dissolve the β-sheet protein, wherein the metallized chelator complex comprises a metal ion chelator and a metal ion.

The invention also provides methods of reducing formation of β-sheet proteins in a subject comprising administering to the subject a metallized chelator complex in an amount sufficient to reduce formation of a β-sheet protein, wherein the metallized chelator complex comprises a metal ion chelator and a metal ion.

The invention further provides isolated metallized chelator complexes comprising a metal ion chelator and a metal ion.

### BRIEF DESCRIPTION OF THE FIGURES

FIG. 1. Raw Fourier transform infrared (FTIR) results for A42 wt dissolved in 1:1 Li⁺ ethylene diamine bromide solution prior to subtraction of the broad vibrational band of heavy water centered at 1550 cm⁻¹, which forms a baseline.

FIG. 2A-2B. FTIR amide I and amide II bands for β-sheet proteins in the solid state and dissolved in 500 mM Cu²⁺ ethylene diamine hydroxide and Li⁺ ethylene diamine bromide chelator/heavy water solutions. A: a) Amide I and II bands for B. mori fibroin (heavy solid line), solid state with curvefitting results for amide I band consisting of four curves (solid lines). b) Amide I and II bands for B. mori fibroin dissolved in Cu²⁺ ethylene diamine hydroxide (dashed line) and Li⁺ ethylene diamine bromide (solid line). B: a) Amide I and II bands for tau paired helical filaments in the solid state (solid line). b) Amide I and II bands for paired helical filaments dissolved in Cu²⁺ ethylene diamine hydroxide (dashed line) and Li⁺ ethylene diamine bromide (heavy solid line).

FIG. 3A-3B. FTIR amide I and II bands for murine prion protein, ME7 isoform. A: Amide I and II bands for the prion protein in the solid state (heavy solid line) with inverted second derivative spectrum (solid line). B: Amide II bands for prion protein ME7 dissolved in the 500 mM Cu²⁺ ethylene diamine hydroxide solution (dashed line). Curve fitting to this result yields two bands at 1651 cm⁻¹ and 1591 cm⁻¹ (solid lines).

FIG. 4A-4B. FTIR amide I and II bands for Aβ 40 wild type and E22Q mutant peptides in the solid state and dissolved in 500 mM Cu²⁺ ethylene diamine hydroxide and Li⁺ ethylene diamine bromide chelator/heavy water solutions. A: a) Amide I band for Aβ 40 wild type (heavy solid line), solid state with curvefitting results consisting of three bands (solid lines). b) Amide I and II bands for Aβ 40 wild type peptide dissolved in Cu²⁺ ethylene diamine hydroxide (dashed line) and Li⁺ ethylene diamine bromide (heavy solid line). Curvefitting for the Li⁺ ethylene diamine bromide Amide I band results, consisting of three bands, is also shown (solid lines). B: a) Amide I and II bands for the Aβ 40 E22Q mutant peptide in the solid state (heavy solid line) with curvefitting results, showing only one of two resultant bands (solid line). b) Amide I and II bands for the Aβ 40 E22Q mutant peptide dissolved in Cu²⁺ ethylene diamine hydroxide (dashed line) and Li⁺ ethylene diamine bromide (heavy solid line). Two of four bands for curvefitting results to the Li⁺ ethylene diamine bromide dissolution amide I peak are also given (solid lines).

FIG. 5A-5B. FTIR amide I and II bands for Aβ 42 wild type and E22Q mutant peptides in the solid state and dissolved in 500 mM Cu²⁺ ethylene diamine hydroxide and Li⁺ ethylene diamine bromide chelator/heavy water solutions. A: a) Amide I and II bands for Aβ 42 wild type (heavy solid line), solid state, with curvefitting results to the Amide I peak consisting of two bands (solid lines). b) Amide I and II bands for Aβ 42 wild type peptide dissolved in Cu²⁺ ethylene diamine hydroxide (dashed line) and Li⁺ ethylene diamine bromide (heavy solid line). Curvefitting for the Li⁺ ethylene diamine bromide results, one band shown (solid line). B: a) Amide I and II bands for the Aβ 42 E22Q mutant peptide in the solid state (heavy solid line) with curvefitting results for the Amide I peak, showing two resultant bands (solid lines). b) Amide I and II bands for the AD 42 E22Q mutant peptide dissolved in Cu²⁺ ethylene diamine hydroxide (dashed line) and Li⁺ ethylene diamine bromide (heavy solid line). Two of four bands for curvefitting results to the Li⁺ ethylene diamine bromide dissolution amide I peak are also given (solid lines).

FIG. 6. Resonance structures for the metallized chelator-protein backbone complex at pH>7. The neutral resonance form is shown at the top, and the dipolar resonance form is given at the bottom. The central sphere represents the Cu²⁺ or Li⁺ cation. The dipolar form maintains the positive charge on the metal ion (Brill et al., 1964).

FIG. 7A-7C. Molecular schematic showing metal chelator square planar geometry for Li⁺ and Cu²⁺ chelated to ethylene diamine in a 1:1 molar ratio at pH>7, and proposed mechanism of β-sheet protein dissolution. A. In a 1:1 molar ratio, the ethylene diamine constitutes one half of the square planar geometric chelation site of either Li⁺ or Cu²⁺. The remaining two planar and two axial chelation sites are occupied by water. B. Deprotonation of β-strand amine groups at pH>7 allows for rotation of the protein backbone about the C—C bond (Coleman and Howitt, 1947). Fragments of β-strands are presented for clarity of presentation. C. Backbone rotation allows deprotonated backbone nitrogens to become available for chelation with the metal cation-ethylene diamine complex, completing the stable, square planar arrangement of ligand bonds about the central cation. Strain-free, pentagonal rings about the central cation are thus formed.

### DETAILED DESCRIPTION OF THE INVENTION

The present invention provides a method of dissolving a β-sheet protein comprising contacting the β-sheet protein with a metallized chelator complex in an amount sufficient to dissolve the β-sheet protein, wherein the metallized chelator complex comprises a metal ion chelator and a metal ion.

As used herein, a “beta-sheet protein” is a protein where hydrogen bonds occur between different polypeptide chains or between separate regions on the same polypeptide chain. The polypeptide chains can run in the same or opposite direction, yielding parallel or anti-parallel structures, respectively.

Metal ions that can be used in the metallized chelator complex include positively charged ions (cations) such as Fe²⁺, Fe³⁺, Zn²⁺, Al²⁺, Ag⁺, Cu⁺, Ni²⁺, Li⁺ and Cu²⁺. Preferred metal ions are one or more of Li⁺ and Cu²⁺.

Metal ion chelators include ethylene diamine, triethylene tetramine HCl, staurosporine aglycone, 4,5-dianilinophthalimide, 1,10-phenanthroline, 1,2-diaminobenzene, derivatives of 1,10-phenanthroline, and derivatives of ethylene diamine, triethylene tetramine HCl, staurosporine aglycone, 4,5-dianilinophthalimide, and 1,2-diaminobenzene except derivatives where the amine hydrogens are substituted. Possible substituents include, but are not limited to, hydroxyl (OH), amine (NH₂), sulfhydryl (SH), the halides (Cl, Br, F, I), methyl (CH₃), ethyl (CH₂CH₃), nitro (NO) and phenyl (C₆H₅). Preferred metal ion chelators are ethylene diamine and triethylene tetramine HCl.

Derivatives of ethylene diamine include compounds with a phenyl substituent at any of positions 1 to 4 indicated below:

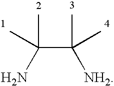

Derivatives of triethylene tetramine dihydrochloride include compounds with a phenyl substituent at any of positions 1 to 12 indicated below:

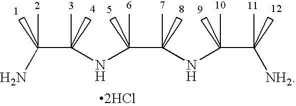

Derivatives of 1,10-phenanthroline include compounds with any of substituents OH, NH₂, SH, Cl, Br, F, I, CH₃, CH₂CH₃, and NO at any of positions 2 to 9 indicated below:

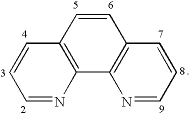

Derivatives of 1,2-diaminobenzene include compounds with any of substituents OH, NH₂, SH, Cl, Br, F, I, CH₃, CH₂CH₃, and NO at any of positions 3, 4, 5 or 6 indicated below:

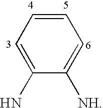

Derivatives of 4,5-dianilinophthalimide include compounds with any of substituents OH, NH₂, SH, Cl, Br, F, I, CH₃, CH₂CH₃, and NO at any of positions 3, 6, 2′ to 6′ and 2″ to 6″ indicated below:

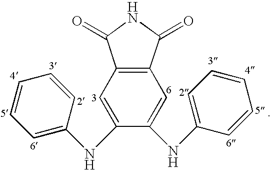

Derivatives of staurosporine aglycone include compounds with any of substituents OH, NH₂, SH, Cl, Br, F, I, CH₃, CH₂CH₃, and NO at any of positions 1 to 5 and 7 to II indicated below:

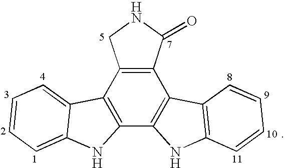

The metal ion and the metal ion chelator can be present in the metallized chelator complex in different molar ratios, for example, in a 1:1 molar ratio or 2:1 molar ratio of metal ion:metal ion chelator, depending on the number of metal binding sites on the chelator. One preferred metallized chelator complex comprises Li⁺ and ethylene diamine in a 1:1 molar ratio. Another preferred metallized chelator complex comprises Cu²⁺ and ethylene diamine in a 1:1 molar ratio. Another preferred metallized chelator complex comprises Lie and triethylene tetramine HCl in a 2:1 molar ratio of Li⁺:triethylene tetramine HCl. Still another preferred metallized chelator complex comprises Cu²⁺ and triethylene tetramine HCl in a 2:1 molar ratio of Cu²⁺:triethylene tetramine HCl.

Preferably, the reaction between the metallized chelator complex and the β-sheet protein is carried out at a pH greater than 7.0.

The metallized chelator complex can bind to different locations on the β-sheet protein, for example, to backbone amides and to histidine residues on the β-sheet protein. Preferably, binding of the metallized chelator complex to the β-sheet protein comprises binding to backbone amides of the β-sheet protein. Preferably, binding between the metallized chelator complex and the β-sheet protein forms a square planar structure.

Preferably, the β-sheet protein is a mammalian β-sheet protein, for example, a sheep, a cow, a steer, a bull, an ox, or a human β-sheet protein.

The β-sheet protein can comprise, for example, a prion protein, a tau protein, a tau paired helical filament, a transthyretin protein, or an amyloid-beta peptide. The β-sheet protein can be associated with a pathological condition, including for example, Alzheimer's disease, Creutzfeldt-Jakob disease, hereditary cerebral amyloid angiopathy, senile systemic amyloidosis, spongiform encephalopathy, Gertsmann-Schenker-Straussler disease, fatal familial insomnia, familial amylotrophic lateral schlerosis, Parkinson's disease or Down syndrome.

The metallized chelator complex can be administered to a subject with a pathological condition characterized by accumulation of beta-sheet solid protein. Preferably, the subject is a mammal. Preferably, the mammal is a human. Preferably, at least one sign or symptom of the pathological condition is improved following administration of the metallized chelator complex to the subject. Symptoms of pathological conditions characterized by accumulation of beta-sheet solid protein include, but are not limited to, inappropriate facial expression, apathy, dizziness, gait abnormality, irritability, weakness in extremities, prominent muscle spasms, blindness, and coma. Basic tests used to assess for dementia include: complete blood count, electrolyte panel, screening metabolic panel, thyroid function tests, vitamin B-12 and folate level check, tests for syphilis and human immunodeficiency antibodies, urinalysis, chest x-ray and electrocardiogram. In the absence of counterindications, brain imaging tests, such as computed tomography and magnetic resonance imaging, can be used to reveal atrophied brain tissue, while an electroencephalogram can be used to reveal abnormal brain wave patterns. Efficacy of the treatment of the subject can be monitored in a variety of ways, for example by improvement in the subject's signs or symptoms or improvement in the subject's score on tests of cognitive function or motor impairment, as appropriate for the subject's specific pathological condition. Memory tests have been developed for assessing memory impairment associated with Alzheimer's disease and other dementias (e.g., U.S. Pat. No. 6,689,058, U.S. Patent Application Publication Nos. 2003/0181793 and 2005/0196735). In addition, beta-amyloid is known to be deposited in the eyes of subjects with Alzheimer's disease (Goldstein et al., 2003). Instruments for monitoring these amyloid deposits are available (Neuroptix Corporation, Acton Mass.). Methods for diagnosing and monitoring Alzheimer's disease through amyloid deposits in the eye have been described (U.S. Pat. No. 6,849,249 B2).

Administration of the metallized chelator complex to a subject can be accompanied by procedures to increase clearance of carbon dioxide from the subject, in order to elevate the subject's pH. Clearance of carbon dioxide can be increased, for example, by having the subject breathe gas with an increased oxygen concentration compared to that of normal air.

The invention further provides a method of preventing or reducing the formation of a β-sheet protein in a subject comprising administering to the subject a metallized chelator complex in an amount sufficient to prevent or reduce formation of the β-sheet protein, wherein the metallized chelator complex comprises any of the metal ion chelators and metal ions disclosed herein. Preferably, the subject is a mammal, for example, a sheep, a cow, a steer, a bull, an ox, or a human.

The metallized chelator complex can be administered to a subject by any convenient route, including but not limited to, oral, subcutaneous, nasal, intravenous, intraperitoneal, intrathecal or intracerebroventricular administration. The dose of metallized chelator complex administered to a subject can be, for example, in the range of 1-100 mg metallized chelator complex/kilogram of body weight/day.

Administration of a metallized chelator complex is believed to have reduced toxicity compared to the toxicity associated with separate administration of metal ions.

The invention also provides an isolated metallized chelator complex comprising a metal ion chelator and a metal ion. Metal ions that can be used include cations such as Fe²⁺, Fe³⁺, Zn²⁺, Al²⁺, Ag⁺, Cu⁺, Ni²⁺, Li⁺ and Cu²⁺. Preferred metal ions are one or more of Li⁺ and Cu²⁺. Metal ion chelators include ethylene diamine, triethylene tetramine HCl, staurosporine aglycone, 4,5-dianilinophthalimide, 1,10-phenanthroline, 1,2-diaminobenzene, derivatives of 1,10-phenanthroline, and derivatives of ethylene diamine, triethylene tetramine HCl, staurosporine aglycone, 4,5-dianilinophthalimide, and 1,2-diaminobenzene except derivatives where the amine hydrogens are substituted. Examples of such derivatives are included in the application. Preferred metal ion chelators are ethylene diamine and triethylene tetramine HCl. The metal ion and the metal ion chelator can be present in the metallized chelator complex in different molar ratios, for example, in a 1:1 molar ratio or 2:1 molar ratio of metal ion:metal ion chelator, depending on the number of metal binding sites on the chelator. One preferred metallized chelator complex comprises Li⁺ and ethylene diamine in a 1:1 molar ratio. Another preferred metallized chelator complex comprises Cu²⁺ and ethylene diamine in a 1:1 molar ratio. Another preferred metallized chelator complex comprises Li⁺ and triethylene tetramine HCl in a 2:1 molar ratio of Li⁺:triethylene tetramine HCl. Still another preferred metallized chelator complex comprises Cu²⁺ and triethylene tetramine HCl in a 2:1 molar ratio of Cu²⁺:triethylene tetramine HCl.

This invention will be better understood from the Experimental Details, which follow. However, one skilled in the art will readily appreciate that the specific methods and results discussed are merely illustrative of the invention as described more fully in the claims that follow thereafter.

### EXPERIMENTAL DETAILS

### Materials and Methods

Reagents: Ethylene diamine, copper hydroxide, lithium bromide and deuterium oxide were purchased from Sigma (St. Louis, Mo.) and used without further purification. Potassium bromide was purchased from ThermoNicolet (Madison, Wis.). Silk worm (Bombyx mori) cocoons were purchased from an artisan fiber supplier, and processed as given below. Amyloid-beta (Aβ) peptides and murine prion protein, ME7 isoform, were a generous gift of Dr. Jorge Ghiso, while the paired helical filaments were generously given by Dr. Peter Davies.

Processing of Raw Silk: The filaments were degummed, i.e. the outer coating of sericin was removed by immersion in a 0.5 M sodium hydroxide solution, followed by dewaxing in several washes of N-hexane (Coleman and Howitt, 1947). This process yields the solid protein fiber, fibroin.

Dissolution of Beta Sheet Proteins by Metal Chelators: 0.5-1.0 mg samples of each peptide and protein were dissolved in 200 μl aliquots of 500 mM heavy water solutions of both 1:1 (molar ratio) Cu²⁺ ethylene diamine hydroxide, pD 13.7 and 1:1 (molar ratio) Li⁺ ethylene diamine bromide, pD 12.8. Each metal salt-chelator solution was prepared with a slight excess of metal salt. Chelator-dissolved proteins were then lyophilized twice to eliminate water and resuspended in 200 μl aliquots of heavy water for spectral analysis.

Fourier Transform Infrared (FTIR) Spectroscopy: Metal chelator-dissolved β-sheet samples were injected into a sample cell with calcium fluoride windows and 0.05 mm path length. The instrument (Nicolet Magna JR 560 Spectrometer, ThermoNicolet, Madison, Wis.) resolution was 4 cm⁻¹; 1000 scans were collected. Spectra of protein-free, blank solutions of Cu²⁺ ethylene diamine hydroxide and 1:1 Li⁺ ethylene diamine bromide and heavy water were also acquired for comparison and baseline subtraction.

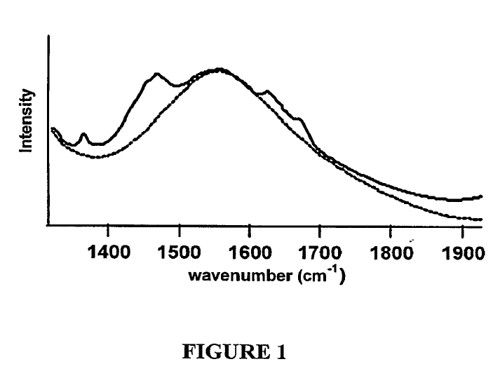

FTIR Spectral Analysis: A broad, heavy water IR band centered at ˜1550 cm⁻¹ (baseline), shown in FIG. 1 along with the raw spectral results for Aβ 42 wt dissolved in 1:1 Li⁺ ethylene diamine bromide heavy water solution, was subtracted from spectral results for all chelator-dissolved samples. Spectral subtraction as well as curve fitting and second derivative analysis of the amide I bands was carried out with the software program, Grams/32 Spectral Notebase, version 4.02, level 1 (ThermoNicolet, Madison, Wis.). Curvefitting parameters were not restricted; the fraction of Lorentzian line shape was allowed to vary. The second derivative analysis was carried out using a Savitsky-Golay algorithm, using either a third or fourth degree polynomial fit.

Mass Spectrometry: Nanoelectrospray ionization was used to obtain the mass to charge ratio (m/z) of ethylene diamine and the 1:1 lithium ethylene diamine complex (˜1.5 mM in water) on a QqTOF mass spectrometer (Applied Biosystems Qstar Pulsar i).

Molecular Models: Molecular diagrams of proposed protein dissolution reactions were constructed using the software program, ChemDraw Std, version 7.0.1 (CambridgeSoft Corp., Cambridge, Mass.).

### Results

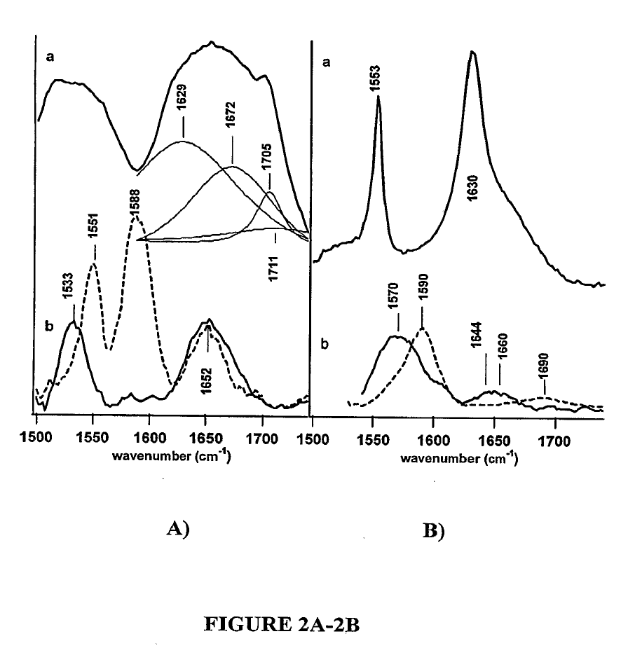

Fibroin. The amide I band frequency is an indicator of peptide and protein secondary structure. The preponderant component of this mode arises from the carbonyl stretch (Krimm and Bandekar, 1986). The amide I frequency, therefore, is sensitive to both intramolecular (α-helix) and intermolecular (β-sheet) hydrogen bonding of the backbone carbonyls. For proteins of high molecular weight, the amide I band is often broad, and can be fit to several constituent bands, reflecting different secondary structural domains. This is the case for the amide I band of the 33 kD B. mori fibroin protein in the solid state (also commonly known as silk), given in FIG. 2A, trace a. Curvefitting results to the broad amide I envelope at 1600-1740 cm⁻¹ yields three peaks at 1629 cm⁻¹, 1672 cm⁻¹ and 1711 cm⁻¹, and one at 1705 cm⁻¹ for the only well-defined peak in the raw data. Second derivative analysis (fourth degree polynomial fit, 15 convolution points), another method of locating peaks in broad, spectral bands, returns peaks at 1616 cm⁻¹, 1651 cm⁻¹ and 1703 cm⁻¹ (Table 1). These values are in general agreement with the infrared dichroism results of Suzuki for fibroin (1967), who found a 1630 cm⁻¹ band perpendicular to the fiber axis, a sharp 1699 cm⁻¹ component parallel to the fiber axis, and a 1656 cm⁻¹ component for both fiber directions. The 1629 cm⁻¹ band is commonly ascribed to β-sheet structure, and both the 1672 cm⁻¹ and 1703-5 cm⁻¹ peaks may be associated with antiparallel chain, pleated β-sheet structure (Krimm and Bandekar, 1986). The amide II band is largely a combination of the inhomogeneous bending mode of the N—H bond and C—N bond stretch modes (Krimm and Bandekar, 1986). Intensity of amide II bands, therefore, is affected by changes in the C—N bond order (Brill et al., 1964; Wang et al., 1921). The amide II band for solid state fibroin (˜1500-1590 cm⁻¹) mirrors the amide I band in its broadness (FIG. 2A, trace a).

Paired Helical Filaments. The Alzheimer's disease-related tau protein is expressed as a set of alternatively spliced protein isoforms. It, too, assumes a fibrillar state. Tau, however, assembles into highly ordered, neuropathological fibers called paired helical filaments that are overwhelmingly β-sheet in structure (Juszczak, 2004). The amide I and II bands for paired helical filaments are shown in FIG. 2B, trace a, solid line. For this protein fiber, the amide I band results are much simpler. It is clear that one band is located at 1630 cm⁻¹, and this is attributed to β-sheet structure (Juszczak, 2004). The amide II band for the paired helical filaments is also very sharp, peaking at 1553 cm⁻¹.

Both of these proteins can be dissolved in basic 500 mM solutions of ethylene diamine chelated in a 1:1 molar ratio to either lithium (Li⁺ ethylene diamine) or copper II (Cu²⁺ ethylene diamine) ions. The dissolution process for the silk or fibroin fiber, readily observable because of its long fiber length, was found to be more rapid—indeed, instantaneous on contact as judged by eye—in the Cu²⁺ ethylene diamine hydroxide solution than in the Li⁺ ethylene diamine bromide solution. The amide I′ and II′ bands for Cu²⁺ ethylene diamine hydroxide/heavy water-dissolved protein are given as dashed lines and the Li⁺ ethylene diamine bromide-dissolved protein, as heavy solid lines in FIGS. 2-5 where spectroscopic results are presented.

The amide I′ bands for fibroin dissolved in both metal ion-ethylene diamine chelator solutions are centered at 1652 cm⁻¹ (FIG. 2A, b), as determined by curvefitting analysis (data not shown); a single band was sufficient to yield a good fit. The broad amide II′ band for solid state fibroin (FIG. 2A, trace a) also collapses to well-defined single peaks at 1551 cm⁻¹ and 1588 cm⁻¹ in the Cu²⁺ ethylene diamine hydroxide dissolved state (FIG. 2A, b, dashed line), and at 1533 cm⁻¹ in the Li⁺ ethylene diamine bromide dissolved state (FIG. 2A, b, solid line). The FTIR results for paired helical filaments dissolved in both metallized chelators are characterized by greatly diminished amide I′ bands, and relatively strong amide II′ bands. The Cu²⁺ ethylene diamine hydroxide-tau solution FTIR result (FIG. 2B, b, dashed line) is dominated by a single amide II′ band at 1590 cm⁻¹, while the Li⁺ ethylene diamine bromide-tau solution result (FIG. 2B, b, heavy solid line) consists of a strong albeit broader amide II′ band centered at 1570 cm⁻¹.

Murine Prion Protein. The prion protein is a 31 kD cellular protein that undergoes a conformational change from a three-helix bundle with a largely unstructured N-terminus to an aggregating β-sheet structure with reduced α-helical content (Peretz et al., 1997). This conformational change results in the formation of cerebral deposits of protein, which are responsible for the neurological disease known as Creutzfeldt-Jakob disease in humans (Prusiner 1997). The disease can be induced in mice, resulting in amyloids of varying molecular structure; one such variant is known as the ME7 isoform.

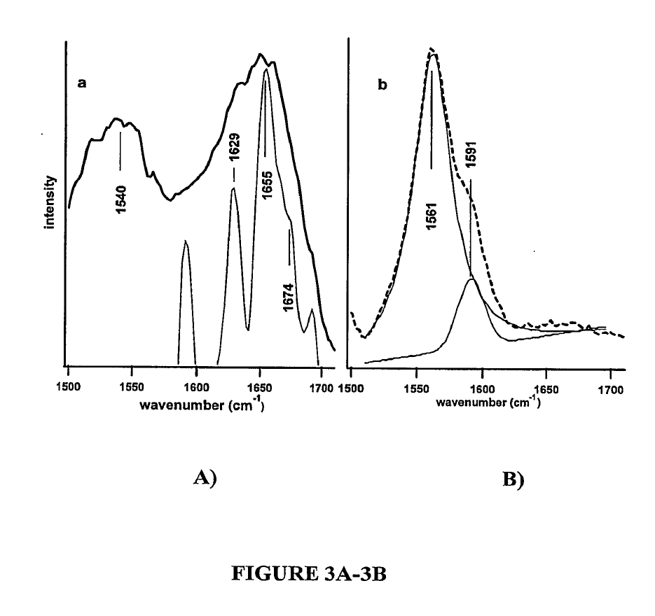

The FTIR amide I and II band results for the murine prion protein ME7 isoform in the solid state, given in FIG. 3A, heavy line, is broad and resolvable into several component bands. Such multicomponent amide bands have been found for solid state PrP 27-30, an N-terminally truncated form of the prion protein, which readily forms amyloid fibrils (Gasset et al., 1993). Second derivative analysis (FIG. 3A, solid line; fourth degree fit, 21 convolution points) yields three major peaks within the amide I band envelope—-shown inverted so that peaks are along the positive y-axis—at 1629 cm⁻¹, 1655 cm⁻¹ and 1674 cm⁻¹.

Dissolution of solid state ME7 prion protein in the 1:1 Cu²⁺ ethylene diamine hydroxide heavy water solution yields FTIR results where the amide I′ band is absent and a very strong amide II′ band is resolvable into a peak at 1561 cm⁻¹ with a shoulder at 1591 cm⁻¹. These results, given in FIG. 3B, parallel those for metallized chelator-dissolved paired helical filaments (FIG. 2B, b).

Aβ Peptides. The Aβ peptides are variable in length—34-42 amino acids in length—and sometimes marked by point mutations that render them pathological (Ghiso et al., 2001). Like tau and the prion protein, they are potentially fibril-forming, and are responsible for several neuropathologies such as Alzheimer's disease and hereditary cerebral amyloid angiopathy-Dutch type (Frangione et al., 2001; Monro et al, 2002; Wisniewski et al., 1991). The specific Aβ peptide responsible for the Dutch hereditary cerebral amyloid angiopathy is 40 residues in length, with a single mutation, E22Q. This single amino acid substitution renders the otherwise water-soluble Aβ 40 wild-type peptide extremely fibrogenic (Wisniewski et al., 1991). In studies of the peptides responsible for the plaques of Alzheimer's disease, however, the longer Aβ 42 predominates (Bush, 2003) and it is this wild-type isoform which is responsible for plaque formation.

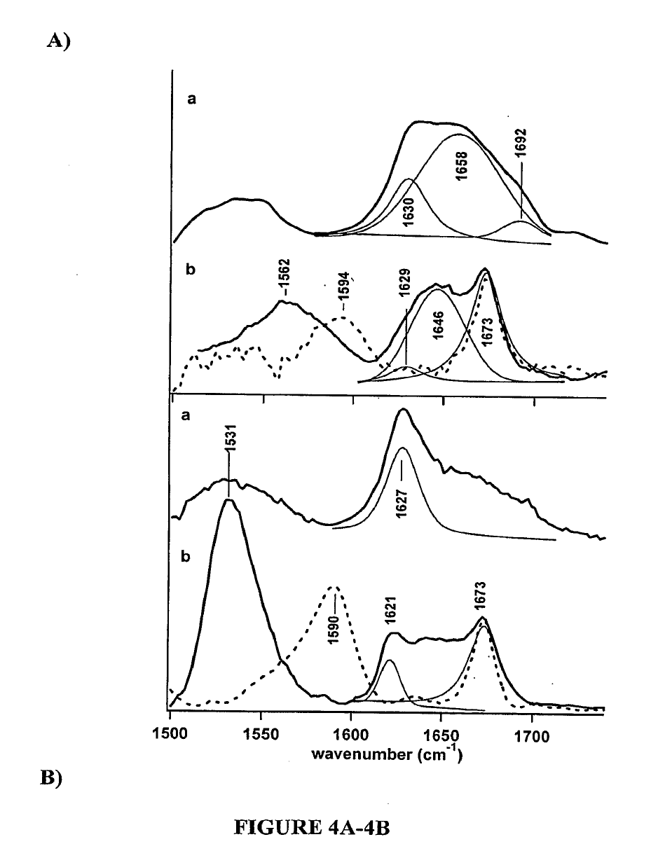

Aβ 40 wild-type and E22Q Mutant. FTIR spectroscopic amide I and amide II band results for the Aβ 40 wild type and E22Q mutant peptides in the solid state and dissolved in 500 mM Cu²⁺ ethylene diamine hydroxide and Li⁺ ethylene diamine bromide/heavy water solutions are presented in FIG. 4. The amide I band for the Aβ 40 wild type peptide (FIG. 4A, a, heavy line) is broad and can be fit to three curves at 1630 cm⁻¹, 1658 cm⁻¹ and 1692 cm⁻¹, shown as solid lines in FIG. 4A, a. Second derivative analysis of this amide I band yielded peaks at 1629 cm⁻¹ and 1661 cm⁻¹ (data not shown). When dissolved in the 1:1 Cu²⁺ ethylene diamine hydroxide solution, the FTIR results for the Aβ 40 wild type peptide yielded a single amide I′ band at 1673 cm⁻¹, shown in FIG. 4A, b, dashed line, and an amide II′ band at 1594 cm⁻¹. Dissolution in the 1:1 Li⁺ ethylene diamine bromide solution resulted in a more complex amide I′ band (FIG. 4A, b, heavy solid line), which was fit to not only a 1673 cm⁻¹ band, but also to 1629 cm⁻¹ and 1646 cm⁻¹ bands, given as solid lines in FIG. 4A, b. The additional amide I′ bands found in the Li⁺ ethylene diamine bromide dissolution results for this peptide—and for others discussed below—are attributed to incomplete deprotonation of peptide amines (Brill et al., 1964).

The amide I band for the Aβ 40 E22Q mutant peptide in the solid state is shown in FIG. 4B, a, heavy solid line. Curvefitting analysis of this result returned two curves: one at 1627 cm⁻¹, shown as a solid line in FIG. 4B, a, and another at 1659 cm⁻¹ (data not shown). The well-defined 1627 cm⁻¹ peak reflects the propensity of the Aβ 40 E22Q mutant to assume a β-sheet structure. As for the Aβ 40 wild type peptide, dissolution of the E22Q mutant in the 1:1 Cu²⁺ ethylene diamine hydroxide solution produced a single amide I′ peak at 1673 cm⁻¹, shown as a dashed line in FIG. 4B, b, and a strong amide II′ peak at 1590 cm⁻¹. Dissolution of the Aβ 40 E22Q mutant in the 1:1 Li⁺ ethylene diamine bromide solution similarly returned a complex amide I′ band (FIG. 4B, b, heavy solid line), but the spectral envelope exhibits greater intensity at the low frequency edge. As for the wild type peptide, curvefit analysis of the amide I′ envelope for the Li⁺ ethylene diamine bromide-dissolved E22Q mutant yields a low wavenumber band at 1621 cm⁻¹ and a high wavenumber band at 1673 cm⁻¹ (FIG. 4B, b, solid lines). The 1621 cm⁻¹ peak clearly represents undissolved β-sheet protein, and amide I′ band area outside of the 1673 cm⁻¹ peak can be attributed to other domains of hydrogen-bonded carbonyls. A very intense amide II′ peak at 1531 cm⁻¹ is associated with the Li⁺ ethylene diamine bromide-dissolved 40 E22Q mutant peptide.

The ability of the metallized chelator, Cu²⁺ ethylene diamine (1:1), to interfere with oligomerization of the neurodegenerative peptide Aβ has been demonstrated through a fibrillization inhibition study of the extremely amyloidogenic Aβ mutant, Aβ 40 E22Q. Oligomerization is the first step in the process of Aβ assembly, leading to fibrillization. Solutions of Aβ 40 E22Q with and without Cu²⁺ ethylene diamine (1:1) were incubated at room temperature for 67 hours at a physiological pH of 7.4. During this time, Aβ 40 E22Q oligomerizes first into multimers, which then reorganize or assemble into fibrils. The fluorescent dye, thioflavin T, was then added to aliquots of each protein solution. The binding of thioflavin T to amyloid fibrils results in a higher fluorescence yield for thioflavin T. This study showed that the fluorescence yield for the Cu²⁺ ethylene diamine-containing aliquot was 27% less than that of the chelator-free aliquot. The conclusion is that the metallized chelator, Cu²⁺ ethylene diamine (1:1) inhibited the oligomerization of the extremely amyloidogenic Aβ40 E22Q and/or interferes with the assembly of oligomers into fibrils at a physiological pH of 7.4. This result is important because it suggests that a metallized chelator drug construct, based on the Cu²⁺ ethylene diamine (1:1) model, can be used as a prophylactic in the early stages of Alzheimer's disease, known as Mild Cognitive Impairment, when the oligomerization process is believed to start.

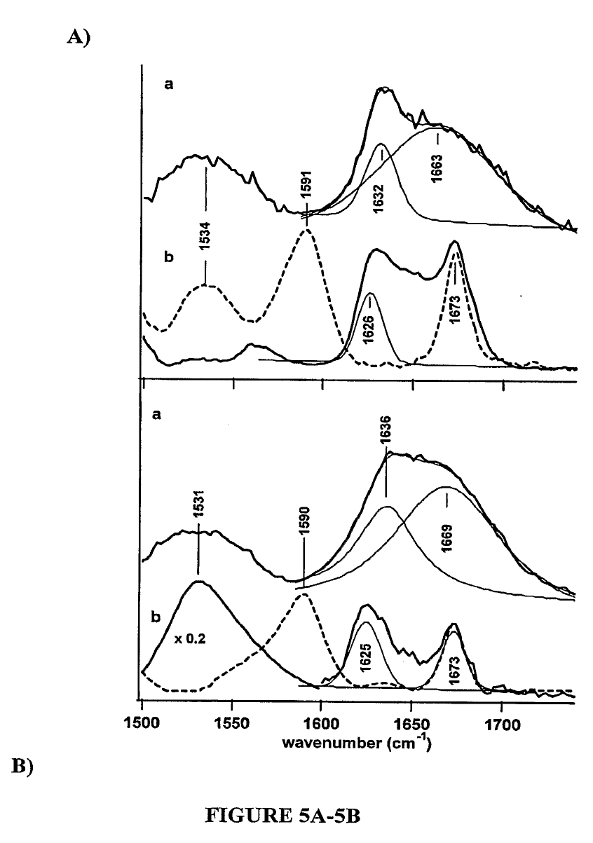

Aβ 42 wild-type and E22Q Mutant. The FTIR amide I and amide II spectral results for the Aβ 42 wild-type and E22Q mutant peptides, given in FIG. 5, can be resolved into a similar pattern of bands. The amide I band for the Aβ 42 wild type peptide in the solid state (FIG. 5A, a, heavy solid line) displays a band shape reminiscent of that for the Aβ 40 E22Q mutant (FIG. 4B, a, heavy solid line) with a prominent low frequency peak at 1632 cm⁻¹, and a broader band at 1663 cm⁻¹. The prominence of the 1632 cm⁻¹ peak reflects the propensity of the Aβ 42 wild type peptide to form a β-sheet structure. On the other hand, the amide I band for the Aβ 42 E22Q mutant in the solid state (FIG. 5B, a, heavy solid line) has no prominent peak, but can be curvefit to bands centered at 1636 cm⁻¹ and 1669 cm⁻¹ (FIG. 5B, a, solid lines) or resolved into peaks at 1636 cm⁻¹, 1671 cm⁻¹ and 1697 cm⁻¹ by second derivative analysis (data not shown). Again, dissolution in the 1:1 Cu²⁺ ethylene diamine hydroxide solution yields a single amide I′ band at 1673 cm⁻¹ for both the Aβ 42 wild type peptide (FIG. 5A, b, heavy dashed line) and the Aβ 42 E22Q mutant (FIG. 5B, b, heavy dashed line) with a strong amide II′ peak at 1590-1 cm⁻¹. Li⁺ ethylene diamine bromide dissolution recapitulates the Aβ 40 peptide pattern of low and high frequency amide I′ peaks at 1625-6 cm⁻¹ and 1673 cm⁻¹, with a connecting region of band area exhibiting no defined peak (FIG. 5A, b, heavy solid line and 5B, b, heavy solid line), and a strong amide II′ peak at 1531 cm⁻¹ for the Aβ 42 E22Q mutant.

Mass Spectrometry of 1:1 Li⁺ ethylene diamine bromide. The calculated mass for protonated ethylene diamine is 61.076572 while that of the 1:1 Li⁺ ethylene diamine bromide complex is 67.084753. The experimentally determined mass-to-charge ratio, m/z, of ethylene diamine and the 1:1 Li⁺ ethylene diamine bromide complex is 61.0843 and 67.0795, respectfully (data not shown). The peak intensity ratio, 67.0795 m/z:61.0843 m/z, is 11.9, supporting the assertion that the 1:1 Li⁺ ethylene diamine bromide complex is formed in an aqueous solution.

TABLE 1
Amide I and II Peaks (cm⁻¹) for Peptides and Proteins in
the Solid and Metal Chelator-Dissolved States Determined by
Analysis of Infrared Spectroscopic Results.

| | AMIDE I (cm⁻¹) | | | | AMIDE II (cm⁻¹) | | |
|---|---|---|---|---|---|---|---|
| Protein | Solid | State | Cu²⁺en | Li⁺en | Solid State | Cu²⁺en | Li⁺en |
| B. mori | 1629^{†} | 1616* | 1652 | 1652 | ~1516-42 | 1551 | 1533 |
| fibroin | 1672 | 1651 | | | | 1588 | |
| | 1705 | 1703 | | | | | |
| | | 1711 | | | | | |
| Paired | 1630 | | — | — | 1553 | 1590 | 1570 |
| helical | | | | — | | | |
| filaments | | | | | | | |
| Murine | | 1629 | — | na | ~1529-49 | 1561 | na |
| prion | | 1655 | | | | 1591 | |
| protein | | 1674 | | | | | |
| Aβ40 wt | 1630 | 1629 | 1673 | 1629 | ~1523-49 | 1594 | 1562 |
| | 1658 | 1661 | | 1646 | | | |
| | 1692 | | | 1673 | | | |
| Aβ40 | 1627 | | 1673 | 1621‡ | ~1522-46 | 1590 | 1531 |
| E22Q | 1659 | | | 1673 | | | |
| Aβ42 wt | 1632 | | 1673 | 1626‡ | ~1522-44 | 1534 | 1562 |
| | 1663 | | | 1673 | | 1591 | |
| Aβ42 | 1636 | 1636 | | 1625‡ | ~1520-39 | 1590 | 1531 |
| E22Q | 1669 | 1671 | 1673 | 1673 | | | |
| | | 1697 | | | | | |

*Values given in italics are for peaks determined by second derivative analysis.
†Values given in nonitalics are for peaks determined by curvefitting analysis.
‡Only the two major curvefit peaks at either edge of the spectral envelope are given.
Cu²⁺en = Cu²⁺ ethylene diamine hydroxide;
Li⁺en = Li⁺ ethylene diamine bromide.

### Discussion

The present application discloses the use of metallized chelators for dissolution of β-sheet proteins. This method is based on the similarity between the ionic radii of copper II (72 pm) and lithium (58 pm), and the complementarity between the pentagonal molecular geometry created in binding of the amine-based chelator, ethylene diamine, and that created when consecutive deprotonated amines from the protein backbone bind (Brill et al., 1964; Freeman, 1967).

The dissolution of the solid state proteins is demonstrated by a comparison of amide I′ and amide II′ absorption bands acquired by FTIR spectroscopy before and after protein dissolution. The FTIR results for metallized chelator-dissolved β-sheet proteins and peptides show similar generalized features: the collapse of a complex amide I and amide II band structure upon dissolution to one or two well-defined bands or—in the case of the amide I band—disappearance of the band including the ˜1630 cm⁻¹ β-sheet marker band; a gain in amide II band intensity at the expense of amide I intensity and the appearance of an amide II′ band at 1590 cm⁻¹. The FTIR solid state and metallized chelator dissolution results for this set of amyloidogenic proteins and peptides are summarized in Table 1.

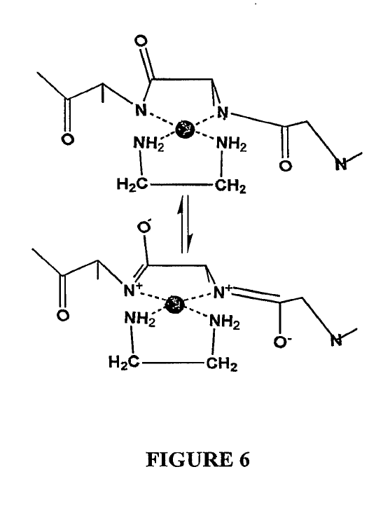

The FTIR results for these β-sheet proteins and peptides arise from a combination of two factors. The deprotonation of the protein backbone nitrogens at pH>7, followed by the binding of the metallized chelator to the nitrogens (Wilson et al., 1971), stabilizes the dipolar resonance form of the resulting complex, shown in FIG. 6 (Brill et al., 1964; Freeman, 1967). In this resonance structure, double bond character is shifted from the carbonyl C—O bond to the backbone C—N bond. The spectroscopic result of this shift is a drastic reduction in the amide I′ band intensity and a concomitant increase in amide II′ band intensity. These changes are most graphically illustrated by the metallized chelator dissolution results for the tau paired helical filaments (FIG. 2B, b) and for the mouse prion protein, ME7 isoform (FIG. 3B), where an amide I′ band is essentially absent. Independent confirmation of these amide I′ and amide I′ band assignment to the dipolar resonance structure is given in the ultraviolet resonance Raman vibrational results for the nonpolar/dipolar resonance structures of N-methylacetamide (Wang et al., 1991). The relative strength of amide I and amide II vibrational bands, which depends upon the population of N-methylacetamide dipolar and nonpolar resonance forms, is controlled by solvent polarity (Wang et al., 1991).

The prediction of a strong amide II′ band at ˜1580 cm⁻¹ for metallized chelator-bound backbone nitrogens (Brill et al., 1964) is realized in all the FTIR results presented here for Cu²⁺ ethylene diamine hydroxide-dissolved amyloids (Table 1). The fact that amide I′ bands appear in the spectroscopic results for fibroin (1652 cm⁻¹, Table 1) and the Aβ peptides (1673 cm⁻¹, Table 1) is attributed to a population of non-chelated backbone nitrogens, and therefore, a population of noncharged carbonyls, arising from nonstoichiometric Cu²⁺ ethylene diamine hydroxide dissolution of protein. This explanation arises from the early dissolution studies of fibroin, where the dissolution mechanism proposed entailed the binding of the Cu²⁺ ethylene diamine hydroxide metallized chelator along the entire length of protein chain, approaching a Cu²⁺:N ratio of 1:2 under alkaline conditions (Coleman and Howitt, 1945, 1947). Thus, in the absence of chelation along the entire protein chain, nonpolar resonance structure sites remain, and an amide I′ band arising from carbonyls can be expected.

This rational can also be extended to the Li⁺ ethylene diamine bromide-dissolution results for the fibroin and tau paired helical filament proteins. The Li⁺ ethylene diamine bromide-dissolution amide I′ band for the Aβ peptides has a second peak at 1621-9 cm⁻¹ resulting from an undissolved β-sheet domain, and unresolved band intensity, arising from other nonchelated backbone domains. At identical concentrations of 500 mM, the more limited dissolution and therefore, chelation to Li⁺ ethylene diamine bromide can be attributed at least in part to the higher alkalinity of the Cu²⁺ ethylene diamine hydroxide solution as the deprotonation of the backbone amines increases with pH (Freeman et al., 1959). Indeed, it has been demonstrated that increasing pH, leading to the deprotonation of amino acid side chains, results in increasing Li⁺ affinity for trimeric metallomacrocycles (Grote et al., 2004). In general, Li⁺ forms weak complexes due to its high solvation energy, and characteristically binds to structurally rigid, small cavity chelators with oxygen ligands (Chang et al., 1995). Yet Li⁺ complexes with nitrogen ligands in an aqueous environment have been reported, and been shown to be quite ionic (Brownstein et al, 1994). It is inferred that the binding constant of Li⁺ for ethylene diamine is lower than that of Cu²⁺.

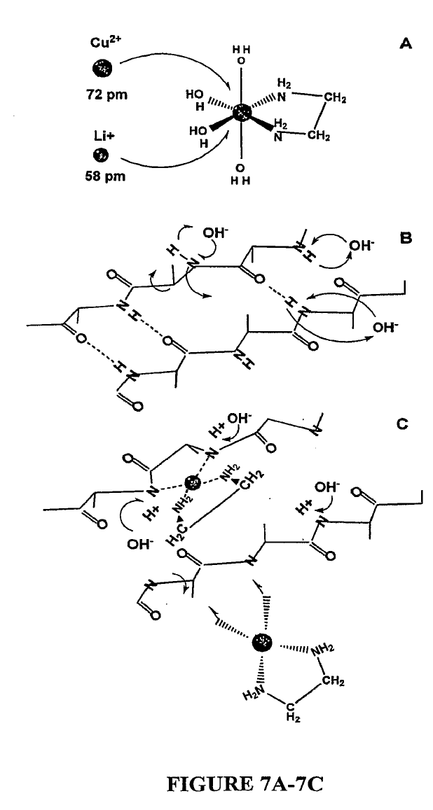

A molecular-level, β-sheet protein dissolution scenario under alkaline conditions and in the presence of the metallized chelators, 1:1 (molar ratio) Li⁺ ethylene diamine bromide or Cu²⁺ ethylene diamine hydroxide, is presented in FIG. 7. The planar, tetragonal arrangement of four of the ligand-field split Cu²⁺ orbitals (FIG. 7A) is consistent with the crystallographic results for the Cu²⁺ complex with biuret (NH₂CONHCONH₂), another bidentate, nitrogen chelator under alkaline conditions (Freeman, 1967; Freeman et al., 1961). Nanoelectrospray ionization mass spectrometry results (data not shown) clearly show the predominance of a 67.10 mass entity in the prepared 1:1 Li⁺ ethylene diamine bromide solution, consistent with the presence of this complex. The square planar geometry characteristic of the Cu²⁺ ethylene diamine complex is also expected for the Li⁺ ethylene diamine bromide complex. The similarity in ionic radius between Cu²⁺ (72 pm) and Li⁺ (58 pm) is an additional rational for the formation of a tetragonal Li⁻ ethylene diamine bromide complex. In the absence of chelator, chelation sites are occupied by water, as shown in FIG. 7A. At pH>7, deprotonation of backbone amines occurs, releasing interstrand hydrogen bonds (Brill et al., 1964; Coleman and Howitt, 1945, 1947; Freeman, 1967), as shown in FIG. 7B. The release of the interstrand hydrogen bonds allows free rotation about the backbone C—C and C—N bonds. In the presence of the metallized chelators, 1:1 Li⁺ ethylene diamine bromide or Cu²⁺ ethylene diamine hydroxide, the backbone deprotonated nitrogens are therefore available for binding to the ligand field orbitals. Thus, solid state β-sheet proteins are dissolved.

The protein fiber, wool, was not found to dissolve in either the Li⁺ ethylene diamine bromide or the Cu²⁺ ethylene diamine hydroxide solution. Wool's tertiary structure is a bundle of α-helices. Insoluble bovine Achilles tendon collagen, type I, was found to be insoluble in Cu²⁺ ethylene diamine hydroxide, but somewhat soluble in Li⁺ ethylene diamine bromide. The polyproline II conformation of collagen places its dihedral angles in the immediate vicinity of those for the β-sheet structure on a Ramachandran plot. Thus, choice of cation confers selectivity.

Chelators alone have shown some success in ameliorating the progression of amyloidogenic neuropathologies where in vivo copper association with the amyloid protein or peptide has been demonstrated (Dedeoglu et al., 2004; Sigurdsson et al., 2003). Trials where mice are sequentially exposed to copper salts followed by treatment with chelator are problematic for two reasons. Where brain pH<7, copper preferentially binds to backbone carbonyls; this follows from earlier studies of the biuret reaction (Brill et al., 1964; Freeman, 1967; Freeman et al., 1961), and thus may actually facilitate interstrand alignment. At pH>7, free copper II forms insoluble copper hydroxide, and so copper is no longer available for chelation.

‘Lithium,’ actually lithium salts such as lithium iodide, have a long history of use in treating psychiatric disorders (Cade, 1949). More recently, lithium salts have been found to reduce the Aβ load in Alzheimer's disease models (Alvarez et al., 1999; Phiel et al., 2003). However, the toxicity associated with lithium salts limits their usefulness in the treatment of diseases such as Alzheimer's disease, where elderly patients are often in general poor health (Alvarez et al., 2002). Lithium bicarbonate has been found to increase lithium influx into human erythrocytes twelve-fold over lithium chloride (Funder et al., 1978). This increased cellular uptake has been attributed to the formation of a Li⁺—CO₃⁻ ion pair with transmembrane transport via the specific anion exchange system (Funder et al., 1978). Looked at another way, it would appear that the CO₃⁻ anion constitutes such a structurally rigid, small cavity chelator, mentioned above, with oxygen ligands at the corners of a trigonal planar molecule, for which Li⁺ has high affinity (Chuang et al., 1995). The results for the LiCO₃⁻ complex suggests that chelation with ethylene diamine may similarly lead to increased Li⁺ uptake, resulting in more effective protein aggregate dissolution. Synergistically, Li⁺ chelation reduces the concentrations required for protein dissolution with the Li⁺ salt alone—from molar units to ˜0.5 molar—as shown here. Thus, more Li⁺ is expected to cross the cell membrane because of chelation, and it is predicted to be more effective in amyloid dissolution than lithium salts alone. Regarding copper ethylene diamine toxicity, the Environmental Protection Agency (1999) has determined that copper-ethylene diamine complex is safe for human consumption.

The metallized chelators, 1:1 (molar ratio) of Li⁺ or Cu²⁺ chelated to ethylene diamine, have been shown here to be potent β-sheet dissolvers. The step of metal cation binding to the ligand prior to in vivo administration of the chelator is believed to ameliorate some of the undesirable consequences of separate administration of metal cation and chelator. For copper, this includes avoidance of insoluble copper II hydroxide precipitation under alkaline conditions and prevention of copper-mediated aggregation under acidic conditions. For lithium, pre-chelation may increase drug efficacy at a lower dosage, and reduce the toxicity associated with lithium salts.

This study also indicates that optimal clearance of amyloid in vivo would benefit from clearance of carbon dioxide to raise physiological pH above neutrality. Although brain pH is buffered, animal studies have shown that a brain pH of 7.2 is achievable (Buxton et al., 1987). On the other hand, chelated copper such as Cu²⁺ ethylene diamine hydroxide can bind to free carbonyls on the protein backbone at pH<7; this follows from the well-studied biuret reaction (Brill et al., 1964; Freeman et al., 1959). As there is evidence of inflammation-induced decrease in brain pH in Alzheimer's disease, these drugs may prove most effective in the early stages of Alzheimer's disease or mild cognitive impairment where interstrand hydrogen bonding is incomplete.

### REFERENCES

Alvarez G, Munoz-Montano J, Satrustegui J, Avila J, Bogonez E, Diaz-Nido J. (1999) Lithium protects cultured neurons against beta-amyloid induced neurodegeneration. FEBS Lett 453:260-264.
Alvarez G, Munoz-Montano J, Satrustegui J, Avila J, Bogonez E, Diaz-Nido J. (2002) Regulation of tau phosphorylation and protection against beta-amyloid-induced neurodegeneration by lithium. Possible implications for Alzheimer's disease. Bipolar Disord 4:153-65.
Brill A, Martin R, Williams R. Copper in biological systems. In: Pullman B, editor. Electronic Aspects of Biochemistry. New York: Academic Press; 1964. p. 519-557.
Brownstein S, Plouffe P-Y, Bensimon C, Tse J. (1994) Complexes of tris((2-pyridyl)alkyl)amines with lithium and sodium salts. Inorg Chem 33:354-8.
Buxton R, Alpert N, Babikian V, Weise S, Coreia J, Ackerman R. (1987) Evaluation of the ¹¹CO₂ positron emission tomographic method for measuring brain pH. I. pH changes measured in states of altered PCO₂. Cereb Blood Flow Metab 7:709-19.
Bush A I. (2003) The metallobiology of Alzheimer's disease. Trends Neurosci 26:207-14.
Cade J. (1949) Lithium salts in the treatment of psychotic excitement. Med J Aust 2:349-52.
Chuang C-L, Frid M, Canary J. (1995) Geometric control of selectivity in alkali metal ion complexation by acyclic N4-tripodal ligands. Tetrahedron Lett 36:2909-12.
Coleman D, Howitt F. (1945) Denaturation and renaturation of proteins. Nature 155:78-9.
Coleman D, Howitt F. (1947) Studies on silk proteins I. The properties and constitution of fibroin. The conversion of fibroin into a water-soluble form and its bearing on the phenomenon of denaturation. Proc Royal Soc A 190:145-69.
Dedeoglu A, Cormier K, Payton S, Tseitlin K, Kremsky J, Lai L, et al. (2004) Preliminary studies of a novel bifunctional metal chelator targeting Alzheimer's amyloidogenesis. Exp Gerontol. 39: 1641-9.
Environmental Protection Agency (1999) Copper-ethylenediamine complex; exemption from the requirement of a tolerance. Fed Register 64:41-44.
Frangione B, Revesz T, Vidal R, Holton J, Lashley T, Houlden H. et al. (2001) Familial cerebral amyloid angiopathy related to stroke and dementia. Amyloid 8, Suppl 1:36-42.
Freeman H. (1967) Crystal structure of metal-peptide complexes. Adv Protein Chem 22:257-424.
Freeman H, Smith J, Taylor J. (1959) Crystallographic studies of the biuret reaction. Nature 184:707-10.
Freeman H, Smith J. Taylor J. (1961) Crystallographic studies of the biuret reaction. I. Potassium bis-biureto cuprate(II) tetrahydrate, K2[Cu(NH2CONHCONH2)2]. 4H2O. Acta Cryst 1961:407-18.
Funder J, Tosteson D, Wieth J. (1978) Effects of bicarbonate on lithium transport in human red cells. J Gen Physiol 71:721-46.
Gasset M, Baldwin M, Fletterick R, Prusiner S. (1993) Perturbation of the secondary structure of the scrapie prion protein under conditions that alter infectivity. Proc Natl Acad Sci USA 90:1-5.
Ghiso J, Revesz T, Holton J, Rostagno A, Lashley T, Houlden H, et al. (2001) Chromosone 13 dementia syndromes as models of neurodegeneration. Amyloid 8:277-84.
Goldstein L E, Muffat J A, Chemy R A, Moir R D, Ericsson M H, Huang X, et al. (2003) Cytosolic beta-amyloid deposition and supranuclear cataracts in lenses from people with Alzheimer's disease. Lancet 361:1258-65.
Grote Z, Scopelliti R, Severin K. (2004) pH-triggered assembly of organometallic receptors for lithium ions. J Am Chem Soc 126:16959-72.
Juszczak L. (2004) Comparative vibrational spectroscopy of intracellular tau and extracellular collagen I reveals parallels of gelation and fibrillar structure. J Biol Chem 279:7395-7404, 18098.
Krimm S, Bandekar J. (1986) Vibrational spectroscopy and conformation of peptides, polypeptides and proteins. Adv Protein Chem 38:181-364.
Monro O, Mackic J, Yamada S, Segal M, Ghiso J, Maurer C, et al. (2002) Substitution at codon 22 reduces clearance of Alzheimer's amyloid-b peptide from the cerebrospinal fluid and prevents its transport from the central nervous system into blood. Neurobiol Aging 23:405-12.
Peretz D, Williamson R, Matsunaga Y, Serban H, Pinilla C, Bastidas R, et al. (1997) A conformational transition at the N-terminus of the prion protein features in formation of the scrapie isoform. J Mol Biol 273:614-622.
Phiel C, Wilson C, Lee V M-Y, Klein P. (2003) GSK-3 alpha regulates production of Alzheimer's disease amyloid-beta peptides. Nature 423:435-9.
Prusiner S. (1997) Prion diseases and the BSE crisis. Science 278:245-51.
Sigurdsson E, Brown D, Alim M, Scholtzova H, Carp R, Meeker H. et al. (2003) Copper chelation delays the onset of prion disease. J Biol Chem 278:46199-202.
Suzuki E. (1967) A quantitative study of the amide I vibrations in the infrared spectrum of silk fibroin. Spectrochim Acta 23A:2303-8.
Wang Y, Purrello R, Georgiou S, Spiro T. (1991) UVRR spectroscopy of the peptide bond. 2. Carbonyl H-bond effects on the ground- and excited-state structures of N-methylacetamide. J Am Chem Soc 113:6368-77.
Wilson E J, Kasperian M, Martin R. (1970) Binding of copper II to potentially tridentate amino acid ligands. J Am Chem Soc 92:5365-72.
Wisniewski T, Ghiso J, Frangione B. (1991) Peptides homologous to the amyloid protein of Alzheimer's disease containing a glutamine for glutamic acid substitution have accelerated amyloid fibril formation. Biochem Biophys Res Comm 179:1247-54.
U.S. Patent Application Publication No. 2003/0181793, published Sep. 25, 2003, Herman Buschke, Memory assessment by retrieval speed and uses thereof.
U.S. Patent Application Publication No. 2005/0196735, published Sep. 8, 2005, Herman Buschke, Memory capacity tests and uses thereof.
U.S. Pat. No. 6,689,058, issued Feb. 10, 2004, Herman Buschke, Memory tests using item-specific weighted memory measurements and uses thereof.
U.S. Pat. No. 6,849,249 B2, issued Feb. 1, 2005, Goldstein et al., Ocular diagnosis of Alzheimer's disease.

1. A method of dissolving a β-sheet protein comprising contacting the β-sheet protein with a metallized chelator complex in an amount sufficient to dissolve the β-sheet protein, wherein the metallized chelator complex comprises a metal ion chelator and a metal ion.

2. The method of claim 1, wherein the metal ion and the metal ion chelator are present in a 1:1 molar ratio or a 2:1 molar ratio of metal ion:metal ion chelator.

3. The method of claim 1, wherein the metal ion is one or more of Fe²⁺, Fe³⁺, Zn²⁺, Al²⁺, Ag⁺, Cu⁺, Ni²⁺, Li⁺ and Cu²⁺.

4. The method of claim 1, wherein the metal ion is one or more of Li⁺ and Cu²⁺.

5. The method of claim 1, wherein the metal ion chelator is ethylene diamine; triethylene tetramine HCl; staurosporine aglycone; 4,5-dianilinophthalimide; 1,10-phenanthroline; 1,2-diaminobenzene; a derivative of 1,10-phenanthroline; or a derivative of ethylene diamine, triethylene tetramine HCl, staurosporine aglycone, 4,5-dianilinophthalimide, or 1,2-diaminobenzene, except a derivative where an amine hydrogen is substituted.

6. The method of claim 1, wherein the metal ion chelator is ethylene diamine or triethylene tetramine HCl.

7. The method of claim 1, wherein the metallized chelator complex comprises Li⁺ and ethylene diamine in a 1:1 molar ratio.

8. The method of claim 1, wherein the metallized chelator complex comprises Cu²⁺ and ethylene diamine in a 1:1 molar ratio.

9. The method of claim 1, wherein the metallized chelator complex comprises Li⁺ and triethylene tetramine HCl in a 2:1 molar ratio of Li⁺:triethylene tetramine HCl.

10. The method of claim 1, wherein the metallized chelator complex comprises Cu²⁺ and triethylene tetramine HCl in a 2:1 molar ratio of Cu²⁺:triethylene tetramine HCl.

11. The method of claim 1, wherein the β-sheet protein is a mammalian β-sheet protein.

12. The method of claim 1, wherein the β-sheet protein is a human β-sheet protein.

13. The method of claim 1, wherein the β-sheet protein comprises a prion protein, a tau protein, a tau paired helical filament, a transthyretin protein, or an amyloid-beta peptide.

14. The method of claim 1, wherein the β-sheet protein is associated with a pathological condition.

15. The method of claim 14, wherein the pathological condition is Alzheimer's disease, Creutzfeldt-Jakob disease, hereditary cerebral amyloid angiopathy, senile systemic amyloidosis, spongiform encephalopathy, Gertsmann-Schenker-Straussler disease, fatal familial insomnia, familial amylotrophic lateral schlerosis, Parkinson's disease or Down syndrome.

16. The method of claim 1, wherein the β-sheet protein is contacted with the metallized chelator complex at a pH greater than 7.0.

17. The method of claim 1, wherein binding of the metallized chelator complex to the β-sheet protein comprises binding to backbone amides of the β-sheet protein.

18. The method of claim 1, wherein binding between the metallized chelator complex and the β-sheet protein forms a square planar structure.

19. The method of claim 1, wherein the metallized chelator complex is administered to a subject with a pathological condition.

20. The method of claim 19, which further comprises increasing clearance of carbon dioxide from the subject.

21. (canceled)

22. A method of reducing the formation of a β-sheet protein in a subject comprising administering to the subject a metallized chelator complex in an amount sufficient to reduce formation of the β-sheet protein, wherein the metallized chelator complex comprises a metal ion chelator and a metal ion.

23-36. (canceled)

37. An isolated metallized chelator complex comprising a metal ion chelator and a metal ion.

38-46. (canceled)
